(19) United States
(12) Patent Application Publication
(10) Pub. No.: US 2023/0414292 A1
(43) Pub. Date: Dec. 28, 2023

(54) REFERENCING APPARATUS

(71) Applicant: Gyder Surgical Pty Ltd, Mulgrave, VIC (AU)

(72) Inventors: Gregory James ROGER, LAGUNA, NSW (AU); William HILL, BURWOOD, VIC (AU); BRENDYN MURRAY RODGERS, BLACKBURN, VIC (AU); Krishna SIVARAMAN, RICHMOND, VIC (AU); Yeap Chin ENG, BALWYN NORTH, VIC (AU)

(21) Appl. No.: 17/800,623

(22) PCT Filed: Feb. 19, 2021
(86) PCT No.: PCT/AU2021/050143

§ 371 Date: Aug. 18, 2022

(30) Foreign Application Priority Data
Feb. 21, 2020 (AU) 2020900504

(51) Int. Cl. A61B 34/20; A61F 2/46

(52) U.S. Cl. CPC A61B 34/20; A61F 2/4609; A61B 2017/00725

(57) ABSTRACT

A referencing apparatus for application onto a patient, the referencing apparatus including: a support plate; a first locating element extending from the support plate; a second locating element extending from the upper support plate; a third locating element extending from the upper support plate; and an electronic orientation monitor housing adapted to receive an electronic orientation monitor; a docking station adapted to receive the housing and thereby define a reference point external to and relative to the patient's anatomy; wherein the first locating element, the second locating element and the third locating element are arranged to be pushed down onto a patient to provide a reference orientation for the electronic orientation monitor with respect to a predetermined anatomical site.

### FIELD OF THE INVENTION

The present invention relates to surgical implements and surgical methods and in particular to a referencing apparatus that may be applied to a patient prior to, or during, surgical procedures, for example hip surgery involving prosthetic components.

### BACKGROUND

The discussion of the prior art within this specification is not, and should not be taken as, an admission of the extent of common general knowledge in the field of the invention. Rather, the discussion of the prior art is provided merely to assist the addressee to understand the invention and is included without prejudice.

Whilst the following discussion is with respect to hip replacement surgery, a person skilled in the art will appreciate that the present invention is not limited to this particular field of use and may be adapted to use with any bone structure or various types of surgery.

Hip replacement surgery involves the use of a prosthetic cup (acetabular cup) or a prosthetic ball (femoral stems) or both to restore the ball and cup joint functionality of the hip. The ball and cup joint enable the hip to rotate in different directions to various degrees (in contrast to the relatively limited rotation of a knee joint).

Historically, hip replacement (arthroplasty) surgery required up to a 40 cm (7 to 12 inches) curved incision to provide sufficient access for the surgeon to manually access and manipulate the hip and femur. A prosthetic cup was attached to the hip socket or the head of the femur removed and replaced with a prosthetic ball, or both.

After the incision is made, the ligaments and muscles are separated to allow the surgeon access to the bones of the hip joint. It is typically this part of the surgery that makes the ligaments and muscles somewhat weak after surgery. Until they heal, which often takes about a month to six weeks, the patient must follow special hip precautions to prevent dislocation of the new hip joint.

Typical steps in hip replacement surgery include the following:
Removing the Femoral Head: Once the hip joint is entered, the femoral head is dislocated from the acetabulum. Then the femoral head is removed by cutting through the femoral neck with a power saw.
Reaming the Acetabulum: After the femoral head is removed, the cartilage is removed from the acetabulum using a power drill and a special reamer. The reamer forms the bone in a hemispherical shape to exactly fit the metal shell of the acetabular component.
Inserting the Acetabular Component: A trial component, which is an exact duplicate of the patient's hip prosthesis, is used to ensure that the joint received will be the right size 5 and fit. Once the right size and shape is determined for the acetabulum, the acetabular component is inserted into place. In the uncemented variety of artificial hip replacement, the metal shell is simply held in place by the tightness of the fit or with screws to hold the metal shell in place. In the cemented variety, a special epoxy type cement is used to “glue” the acetabular component to the bone.
Preparing the Femoral Canal. To begin replacing the femoral head, special rasps are used to shape and hollow out the femur to the exact shape of the metal stein of the femoral component. Once again, a trial component is used to ensure the correct size and shape. The surgeon will also test the movement of the hip joint.
Inserting the Femoral Stem: Once the size and shape of the canal exactly fit the femoral component, the stem is inserted into the femoral canal. Again, in the uncemented variety of femoral component the stem is held in place by the tightness of the fit into the bone (similar to the friction that holds a nail driven into a hole drilled into wooden board—with a slightly smaller diameter than the nail). In the cemented variety, the femoral canal is rasped to a size slightly larger than the femoral stem. Then the epoxy type cement is used to bond the metal stem to the bone.
Attaching the Femoral Head. The metal ball that replaces the femoral head is attached to the femoral stem.
The Completed Hip Replacement: Before the incision is closed, an x-ray is taken to make sure the new prosthesis is in the correct position.

Such surgery had a number of problems including:
a hospital stay of three days or more, post-operative pain and weeks of rehabilitation;
each cm of incision has a tenfold increase in the risks of blood clotting and infection post-surgery;
the surgeon was reliant on his experience and eye to ensure accurate placement of the cup into the three dimensional hip socket and alignment of the cup with the ball/femur to enable proper function of the joint. Misalignment may lead to post-operative complication such as misalignment of the leg, incorrect leg length and/or incorrect soft tissue tension. The long-term effects of misaligned prosthetic components can also include accelerated wear of the components, aseptic loosening of the components and potentially early repetition of the surgery.

Attempts to overcome these problems include:
WO 2003/037192 which discloses a jib (impaction tool) for use in bone surgery and thus enables the use of a smaller incision. For hip replacement surgery, the jig enables the use of a 4 to 7 cm (2 to 3 inch) incision, i.e. keyhole surgery. Other benefits include a shorter stay in hospital, less blood loss, less pain, fewer postoperative dislocations and faster recovery; and
WO 2005/046475 which discloses a gauge to assist the surgeon with accurate placement of a prosthetic when using a jig in keyhole surgery as the surgeon is no longer able to see the fit of the cup into the hip socket or the fit between the ball and cup.
WO 2010/031111 which discloses a brace designed to define a reference point with respect to the patient in use to define the location for prosthetic placement.

The gauge provided in WO 2005/046475 has enabled efficient use of the impaction tool of WO 2003/037192. Commercial examples include the NivNav Hip System available from MAC Surgical. However, the gauge only works in two dimensions and there is still a heavy reliance on the surgeon's eye and experience for optimal placement of the cup into the hip.

A further attempt to overcome these problems was provided by WO 2010/031111, the contents of which are hereby incorporated in their entirety into this specification by way of cross reference. This prior art document discloses a brace (3) in the form of a clamp that is attachable to a patient to define a reference point relative to the patient's anatomy. This prior art clamp has a number of pads (14, 16, 17 and 18) that are positioned against various points of the patent's anatomy. However, it has been appreciated by the present inventor that this clamp is likely to be obstructive to at least some surgical procedures and does not readily accommodate the surgical drapes that are typically used in many surgical settings.

### SUMMARY OF THE INVENTION

It is an object of the present invention to overcome or ameliorate at least one or more of the disadvantages of the prior art, or to provide a useful alternative.

According to a first aspect of the invention, there is provided a referencing apparatus for application onto a patient. The referencing apparatus may include a support plate. The referencing apparatus may further include a first locating element extending from the support plate. The referencing apparatus may further include a second locating element extending from the upper support plate. The referencing apparatus may further include a third locating element extending from the upper support plate. The referencing apparatus may further include an electronic orientation monitor housing adapted to receive an electronic orientation monitor. The referencing apparatus may further include a docking station adapted to receive the housing and thereby define a reference point external to and relative to the patient's anatomy. The first locating element, the second locating element and the third locating element may be arranged to be pushed down onto a patient to provide a reference orientation for the electronic orientation monitor with respect to a predetermined anatomical site.

According to a particular arrangement of the first aspect, there is provided a referencing apparatus for application onto a patient, the referencing apparatus including: a support plate; a first locating element extending from the support plate; a second locating element extending from the upper support plate; a third locating element extending from the upper support plate; and an electronic orientation monitor housing adapted to receive an electronic orientation monitor; a docking station adapted to receive the housing and thereby define a reference point external to and relative to the patient's anatomy; wherein the first locating element, the second locating element and the third locating element are arranged to be pushed down onto a patient to provide a reference orientation for the electronic orientation monitor with respect to a predetermined anatomical site.

The housing may be a sterile housing. The housing may comprise: a body comprising an opening adapted for receiving the orientation monitor; a load funnel comprising an angled, leading wall, and an insertion wall for enabling a non-sterile orientation monitor to be inserted into the sterile housing whilst preventing contamination of the sterile housing; and a locking means comprising an enclosed frame adapted for engagement with the docking station, wherein the frame is pivotally attached to the body of the housing. The housing may be a clamshell housing.

The load funnel may be a transfer shield adapted to be placed over the opening of the housing to protect the sterile components of housing in use whilst the monitor is inserted through the shield and into the housing.

The locking means may comprise a central aperture shaped to receive the docking station within it, thereby to fix the orientation monitor to the reference apparatus to orient the electric orientation monitor with respect to the patient.

The first and second locating elements may be first and second anterior superior iliac spine locating elements. The third locating element may be a pubis locating element. The first and second locating elements may include a patient connecting plate. The patient connecting plate maybe arranged to fix to a patient above the anterior superior iliac spine.

An electronic orientation monitor may be dockable with the docking station in either a first docking configuration or a second docking configuration. The electronic orientation monitor may be housed in an electronic orientation monitor housing. The electronic orientation monitor housing may be arranged to engage the support plate to provide the reference orientation for the electronic orientation monitor.

The first docking configuration may define a first orientation of the electronic orientation monitor relative to the referencing apparatus. The second docking configuration may define a second orientation of the electronic orientation monitor relative to the referencing apparatus.

According to a second aspect of the invention, there is provided a method of using a referencing apparatus as defined in the first aspect to calibrate an electronic orientation monitor. The method may include the step of positioning a patient connector plate over the left anterior superior iliac spine. The method may include the further step of positioning a patient connector plate over the right anterior superior iliac spine. The method may include the further step of connecting the first and second locating elements to respective patient connector plates. The method' may include the further step of positioning the third locating element against the pubis. The method may include the further step of placing an electronic orientation monitor into a sterile housing comprising means for receiving the non-sterile monitor whilst preventing contamination with the sterile housing. The method may include the further step of engaging the housing comprising the monitor housed therein to a docking station disposed on the referencing apparatus to assume a reference orientation external to and relative to the patient. The method may include the further step of calibrating the electronic orientation monitor.

According to a particular arrangement of the second aspect, there is provided a method of using a referencing apparatus as defined in the first aspect, to calibrate an electronic orientation monitor, the method including the steps of: positioning a patient connector plate over the left anterior superior iliac spine; positioning a patient connector plate over the right anterior superior iliac spine; connecting the first and second locating elements to respective patient connector plates; positioning the third locating element against the pubis; placing an electronic orientation monitor into a sterile housing comprising means for receiving the non-sterile monitor whilst preventing contamination with the sterile housing; engaging the housing comprising the monitor housed therein to a docking station disposed on the referencing apparatus to assume a reference orientation external to and relative to the patient; and calibrating the electronic orientation monitor.

The referencing apparatus may be disposed anterior to the patient during the steps of pressing the referencing apparatus into engagement with the surgical drapes and during calibration of the electronic orientation monitor. An anterior approach may be used for surgical access to the patent's acetabulum.

The patient may be positioned such that they lie in a face up position during the above method steps.

According to a third aspect of the invention, there is provided a method of using a referencing apparatus as defined in the first aspect, to calibrate an electronic orientation monitor. The method may include the step of positioning surgical drapes onto a patient. The method may include the further step of engaging the plurality of locating elements against the surgical drapes such that the surgical drapes are disposed intermediate the plurality of locating elements and a respective plurality of predefined anatomical sites on the patient so as to cause a docking station disposed on the referencing apparatus to assume a reference orientation relative to the plurality of predefined anatomical sites. The method may include the further step of placing an electronic orientation monitor into a sterile housing comprising means for receiving the non-sterile monitor whilst preventing contamination with the sterile housing and docking the housing with the docking station so as to orient the electronic orientation monitor in a reference orientation. The method may include the further step of calibrating the electronic orientation monitor.

According to a particular arrangement of the third aspect, there is provided a method of using a referencing apparatus as defined in the first aspect, to calibrate an electronic orientation monitor, the method including the steps of: positioning surgical drapes onto a patient; engaging the plurality of locating elements against the surgical drapes such that the surgical drapes are disposed intermediate the plurality of locating elements and a respective plurality of predefined anatomical sites on the patient so as to cause a docking station disposed on the referencing apparatus to assume a reference orientation relative to the plurality of predefined anatomical sites; placing an electronic orientation monitor into a sterile housing comprising means for receiving the non-sterile monitor whilst preventing contamination with the sterile housing and docking the housing with the docking station so as to orient the electronic orientation monitor in a reference orientation; and calibrating the electronic orientation monitor.

### BRIEF DESCRIPTION OF THE DRAWINGS

Embodiments of the invention will now be described, by way of example only, with reference to the accompanying drawings, in which:

FIG. 1 is a perspective view of a referencing apparatus according to a first embodiment of the present invention;

FIG. 2 is a perspective view of a referencing apparatus of the embodiment of FIG. 1;

FIG. 2A is a perspective view of a further embodiment of a referencing apparatus;

FIG. 2B is a perspective view of a further embodiment of a referencing apparatus;

FIG. 3 is a partial perspective view of a first or second locating element used with the referencing apparatus of FIG. 1;

FIG. 4 is cross sectional view of the first or second locating element of FIG. 3;

FIG. 5 is an exploded view of an electronic orientation monitor used with the reference apparatus of FIG. 1;

FIGS. 5A and 5B show an alternative embodiment of the electronic orientation monitor housing including a clamshell case and transfer shield; FIG. 5;

FIG. 5C is an exploded view of the alternative electronic orientation monitor of FIG. 5A;

FIG. 6 is a perspective view of a clamshell case for the electronic orientation monitor of

FIG. 7 is a side view of the clamshell case of FIG. 6; and

FIGS. 8A and 8B shows the clamshell housing engaged with an embodiment of the referencing apparatus of FIG. 1;

FIG. 9 is a perspective view of the electronic orientation monitor of FIG. 5 attached to a surgical impactor.

### DETAILED DESCRIPTION OF THE PREFERRED EMBODIMENTS

Broadly, the present invention relates to a referencing apparatus 100 that is for application onto a patient for hip surgery such as, but not limited to, a hip replacement. Referring to the drawings, the referencing apparatus 100 of the present invention is used for assisting a surgeon to orient a prosthetic component relative to a patient's anatomy during surgery. The referencing apparatus 100 is able to be placed over the patient by a surgeon allowing for the easy and reliable orientation of an electronic orientation monitor 46 in housing 12. When oriented, the electronic orientation monitor can be disengaged from the referencing apparatus 100 and attached to the prosthetic component to provide position details of the prosthetic component for reliable and accurate insertion of the prosthetic component into the patient. The prosthetic component may, for example, be a surgical impactor which is used to place the prosthetic component into the patient. The orientation monitor provides position details of the impactor which is equivalent to the position of the prosthetic to ensure accurate placement of the prosthetic component within the patient orientation monitor 46 comprises an inertial measurement unit (IMU) including a tri-axial digital gyroscope and tri-axial digital accelerometer. In particular embodiments, the IMU is an integrated digital gyroscope and accelerometer sensor specifically designed for inertial measurements and provides increased dynamic range, improved sensitivity, greater accuracy, lower bias and lower drift when compared with prior gyroscope/accelerometer implementations. In particular embodiments, the IMU selected enables precise positional awareness and determination without the need for additional navigational components, such as, for example, magnetometer or compass components which may experience interference from localised magnetic fields (e.g. from medical imaging equipment) which would adversely affect the accuracy of the critical orientation parameters which must be determined by the orientation monitor 46. In particular embodiments, the surgeon may be provided with the ability to select or enter specific prosthetic angles and related data to the orientation monitor 46 to assist and fine tune the orientation calibration with respect to the patient's unique anatomy.

The preferred embodiment is particularly suited for assisting surgeons to properly locate an acetabular cup into a reamed acetabulum during hip surgery, such as total or partial hip replacements or revisions. When used in this context, the reference apparatus 100 is pushed down from above onto the patient's pelvis to assume a reference position to orient an electronic orientation monitor 46 to assist in the positioning of surgical impactors and prosthetic components.

This detailed description shall describe the use of the referencing apparatus 100 and electronic orientation monitor 46 as an aid in the insertion of a prosthetic component in the form of an acetabular cup, into a reamed acetabulum of a patient's pelvis wherein an anterior approach is used for surgical access to the patent's acetabulum. An important part of this process is the use of the referencing apparatus 100 to calibrate an electronic orientation monitor 46, which may be similar to the monitor as disclosed in applicant's prior application no. WO 2010/031111, the contents of which have been incorporated in their entirety into this specification by way of cross reference. However, it will be appreciated by those skilled in the art that the invention may be used in other surgical contexts.

With reference to the Figures, the referencing apparatus 100 comprises the following components:
a frame 10, for positioning of the electronic orientation monitor in a reference position with respect to the patient;
first and second anterior superior iliac spine (ASIS) locating elements 14;
a third, pubis locating element 16;
an electronic orientation monitor 46; and
electronic orientation monitor housing 12.

The frame 10 includes an upper support plate in the form of channel section 11 and T channel

section 15 extending perpendicularly from the channel section 11. A first ASIS locating element 14 extends outwardly from the base of one longitudinal end of the channel section 11 and the second ASIS locating element 14 extends from the opposite longitudinal end of the channel section 11. The third, pubis locating element 16 extends outwardly from the base of the T channel section 15.

A force applying plate 13 forms part of the channel section 11 and provides an interface for downwards force to be applied to the referencing apparatus 100 onto the patient through the locating elements 14, 16 to orient the electronic orientation monitor 46.

The first and second ASIS locating elements 14 connect to the channel section 11 and extend outwards and generally perpendicularly to the channel section 11. In one embodiment, to provide the ability to use a single sized referencing apparatus 100 on different sized and hip profile patients, the first and second ASIS locating elements 14 attach to the channel section 11 so that they can be fixed at different positions. In one embodiment, the connecting face of the channel section 11 has a number of fixing points that can be selected from to attach the first and second ASIS locating elements 14. In an alternative embodiment the underside of the channel section 11 includes a rail with an attachment point for the first and second ASIS locating elements 14. The attachment point can be locked in place as desired.

The third, pubis locating element 16 connects to the T channel section 15 and extend outwards and generally perpendicularly to the T channel section 15. In one embodiment, to provide the ability to use a single sized referencing apparatus 100 on different sized and hip profile patients, the third, pubis locating element 16 is attached to the T channel section 15 so that it can be fixed at different positions. In one embodiment, the connecting face of the T channel section 15 has a number of fixing points that can be selected from to attach the third, pubis locating element 16.

In an alternative embodiment the underside of the T channel section 15 includes a rail with an attachment point for the third, pubis locating element 16. The attachment point can be locked in place as desired.

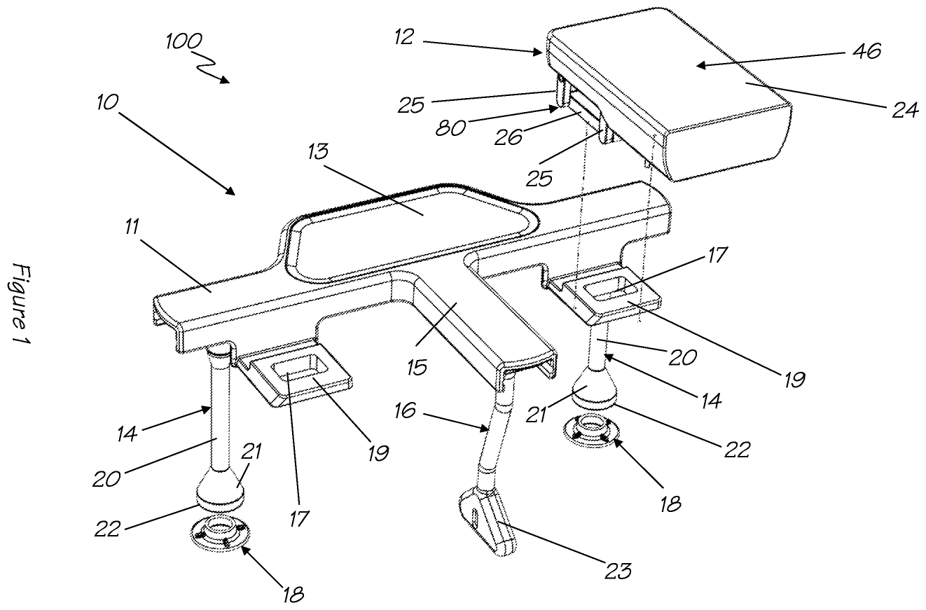

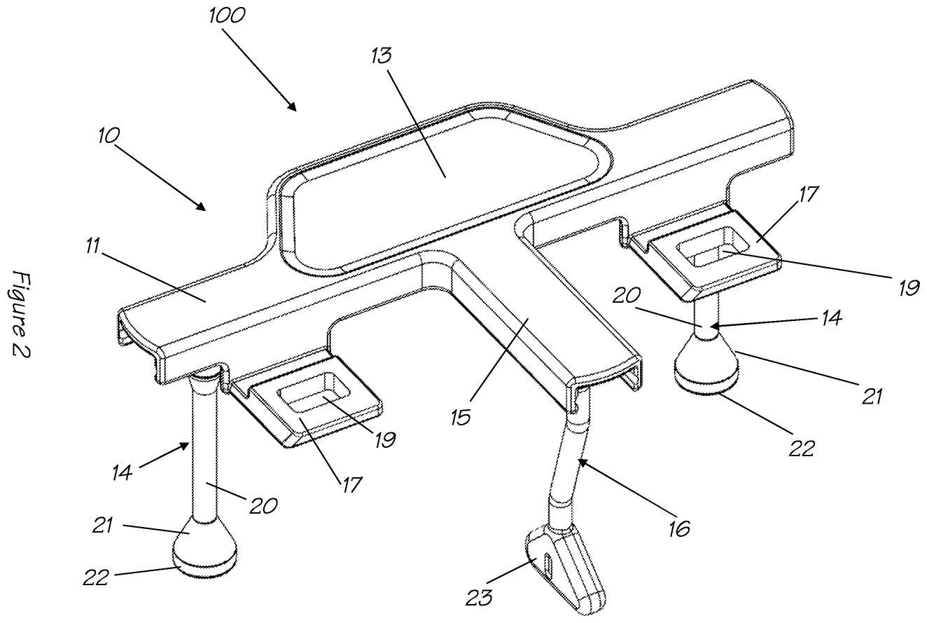

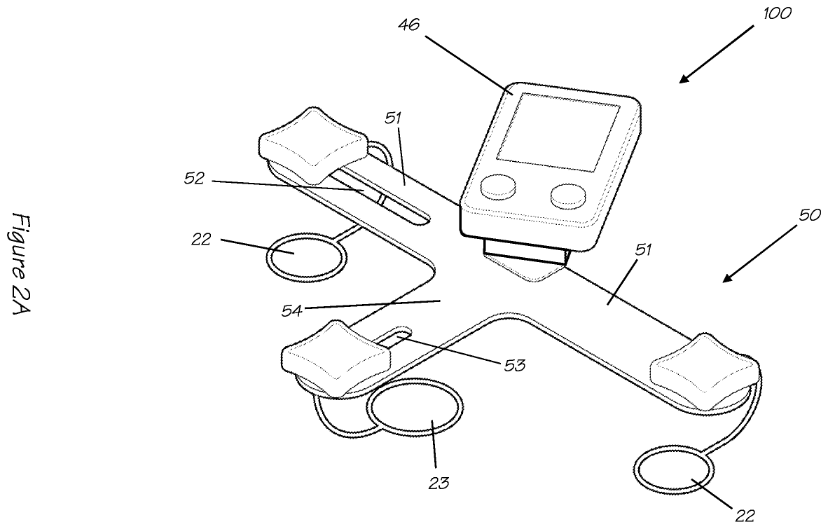

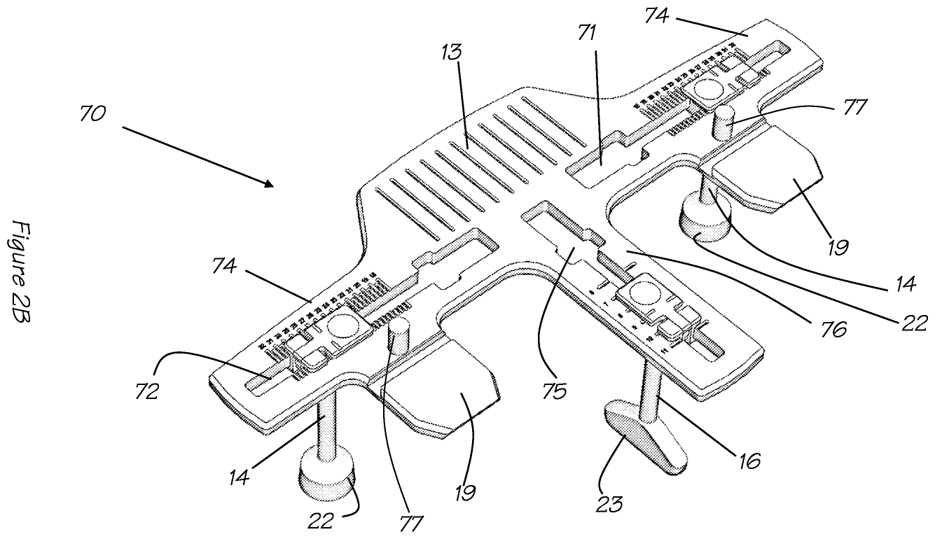

Extending from the channel section 11 are docking stations 19 shaped to receive the electronic orientation monitor 46 contained within the housing 12. Docking stations 19 are designed to be highly toleranced and locate the monitor 46 in a precisely defined position so as not to compromise the accuracy of the measuring system. Engaging either one of the docking stations 19 with the electronic orientation monitor 46 contained within the housing 12 defines a reference point relative to the patient's anatomy. This reference point is external of the patient and is used to orient the electronic orientation monitor 46 into a reference orientation. Whilst in this orientation the electronic orientation monitor 46 acquires reference orientation information which is used to calibrate electronic orientation monitor 46 to the reference orientation. In one embodiment, as shown in in FIG. 1, the docking station 19 of frame 10 includes a central aperture 17 and the docking station 19 is profiled to receive and fix in place the electronic orientation monitor 46. In further embodiments of the frame, e.g. frame 70 of FIG. 2B, docking stations 19 do not comprise an aperture therein.

First and second ASIS locating elements 14 include a rod extension 20 that extends from the base of the channel section 11, a base 21 and patient connecting plate 18. The patient connecting plate 18 is arranged to be fixed to the patient's body over the patient's ASIS. The position of the patient connection plate 18 defines where the first and second ASIS locating elements 14 will be placed on the patient.

The pubis locating element 16 extends as a bar from the channel section 11 and ends in pubis probe 23. The pubis probe 23 is shaped to nestle within the ridges of the pubis of the patient in use. With the pubis probe 23 nestled between the pubis ridges on the patient and the first and second ASIS locating elements 14 in place on the connection plates 18, a known reference position of the channel section 11 and referencing apparatus 100 as a whole, with reference to the patient, is then known.

In this known position, the electronic orientation monitor 46 can be placed on the docking station 19 in the electronic orientation monitor housing 12 appropriate for the hip side that they are to operate on in accordance with the surgeon's personal preference i.e. regardless of the hip which the surgeon in to operate on, the surgeon may place the orientation monitor 46 in housing 12 and attach the housing to either of the two docking stations 19 (on either the left or right side of frame 10, 70) of the referencing apparatus 100. In practice, the range of desired locations selectable by the surgeon for the orientation monitor 46 is limited by the anatomy of the patient and clinical efficacy. The location selected by the surgeon is determined by a combination of the type of implant and the patient's anatomy tempered by the surgeon's clinical judgement. The referencing apparatus 100 can be considered to establish a commonly used/accepted pre-defined orientation with respect to the patient's anatomy. The addition of the orientation monitor 46 to the apparatus 100 provides the surgeon with live positional feedback, which enables the surgeon to position the implant in a desired location which may differ from the pre-defined position, should the surgeon think this appropriate.

FIG. 2A shows an alternate embodiment 50 of frame 10 wherein channel section 11 and T-channel section 15 of the prior embodiment are replaced with rigid non-channel arms 51 and 52 respectively are replaced with a non-channel frame 50. Adjustment slots 52 and 53 are provided in at least one of arms 51, and also in T-section 54 for adjustment of the spacing between the first and second ASIS locating elements 14 having feet 22, and also for adjustment of pubis locating element 16 with respect to the patient's unique anatomy. The particular embodiment show in FIG. 2A is described in greater detail in Australian Patent No. AU2013204941, the contents of which are entirely incorporated herein by cross reference.

FIG. 2B shows an alternate embodiment 70 of frame 10 and frame 50 wherein, again a channel section frame is not used but rather slots 71 and 72 on the arms 74 of frame 70 and also, slot 75 on T-section 76 (similar in nature to slots 52 and 53 of FIG. 2A) are provided. In this present embodiment, ASIS locating elements 14 terminate in a lever lock system which engages with slots 71 and 72 respectively of frame 70. Similarly, pubis locating element 16 terminates in a lever lock system which engages with slot 75 so that the ASIS locating elements 14 and pubis locating element 16 can be adjusted to locate the anatomical features of the patient and then be locked in position. As shown in FIG. 2A, slots 71 and 72 of arms 74 and slot 75 of T-section 76 may optionally comprises a series of regularly spaced detents to assist in correctly locating the locating elements 14 and 16 and also to aid the locking clamps to secure the locating elements 14 and 16 in place.

Docking stations 19 of the embodiment shown in FIG. 2B also comprises magnet locating features 77. Locating features 77 are sensed by the orientation monitor 46 when engaged with either of the docking stations 19. Locating features 77 sense placement of the orientation monitor housing 12 including the orientation monitor 46 and assist in ensuring that referencing apparatus 100 is used as intended and to avoid error, for example, which may arise if the monitor 46 or the frame 10, 50, 70, is used before it has been properly calibrated.

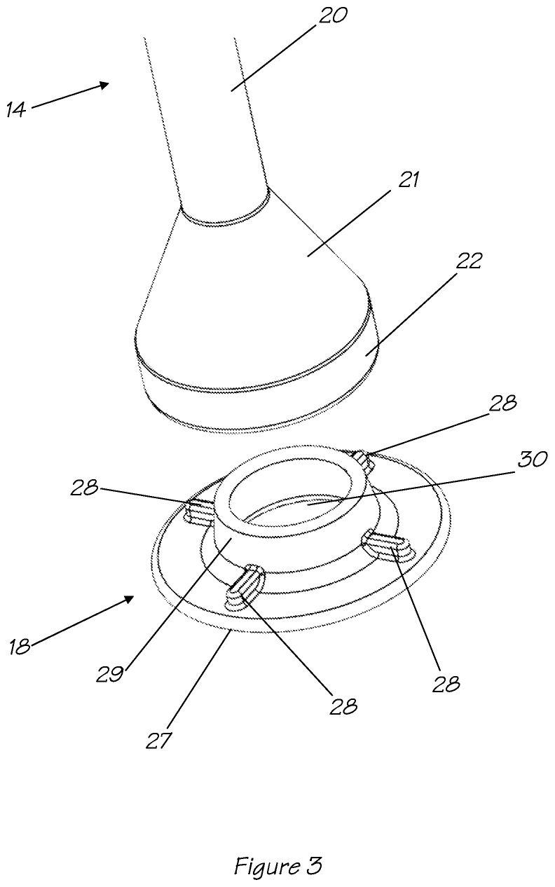

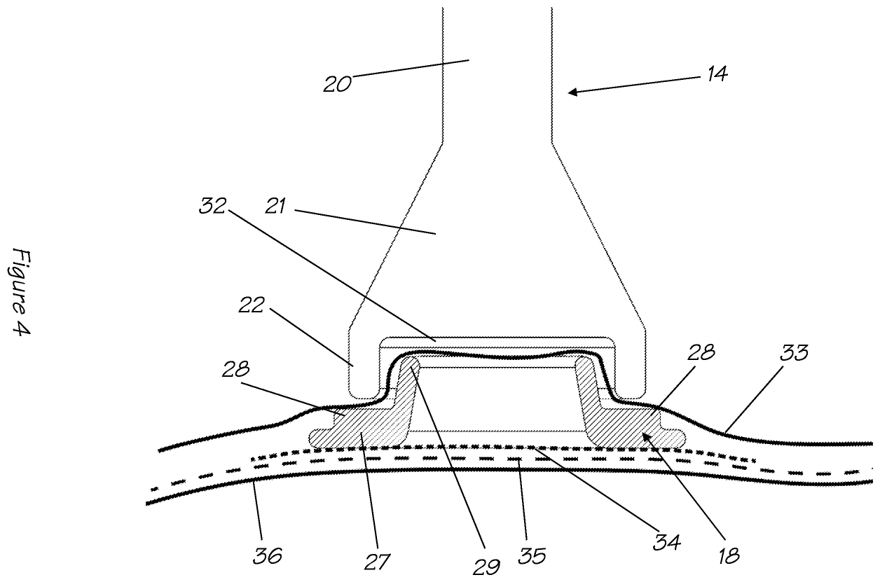

As best shown in FIGS. 3 and 4, the patient connecting plate 18 includes a central ring protrusion 29 defined by the perimeter 22 of the first and second ASIS locating elements 14, a central aperture 30 defined by the central ring protrusion 29, a base 27 and base protrusions 28. The central ring protrusion 29 acts to be received in the recess 32 defined by the perimeter 22 of the first and second ASIS locating elements 14. With a patient connecting plate 18 fixed in place on both the left and right second ASIS, the recesses 32 on first and second ASIS locating elements 14 can be placed over the central ring protrusions to place the referencing apparatus 100 in a reference orientation when the pubis locating element 16 is also in place. The ring structure of patient connecting plate 18 also allows the surgeon to easily place the patient connecting plate 18 directly over the patient's ASIS when the patient is covered by surgical drapes

FIG. 4 illustrates the fixing of the patient connecting plate 18 to the patient. An adhesive sterile film 35 is attached to the patient's skin 36. A connector plate adhesive 34 is stuck to the adhesive film 35 over the anterior superior iliac spine and fixes external of the patient and is used to orient the electronic orientation monitor 46 into a reference orientation. Whilst in this orientation the electronic orientation monitor 46 acquires reference orientation information which is used to calibrate electronic orientation monitor 46 to the reference orientation. A patient covering sheet 33 can be accommodated between the patient connecting plate 18 and the first and second anterior superior iliac spine locating elements 14.

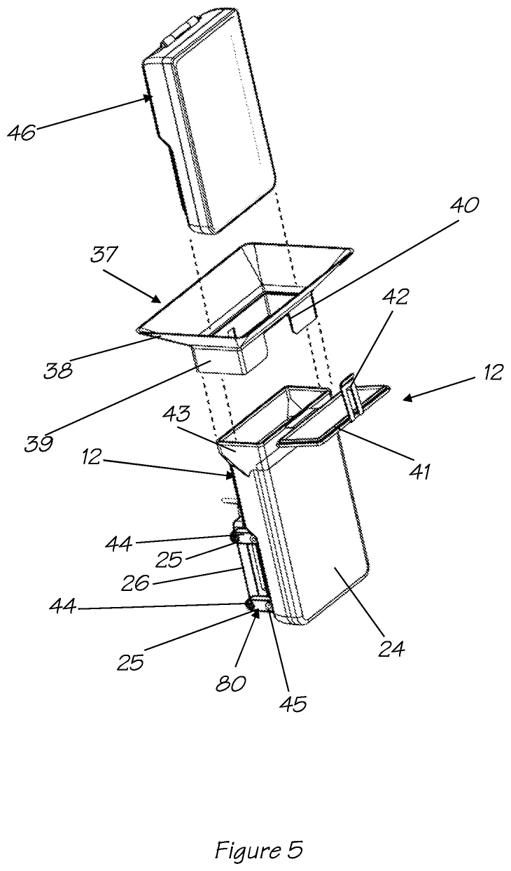

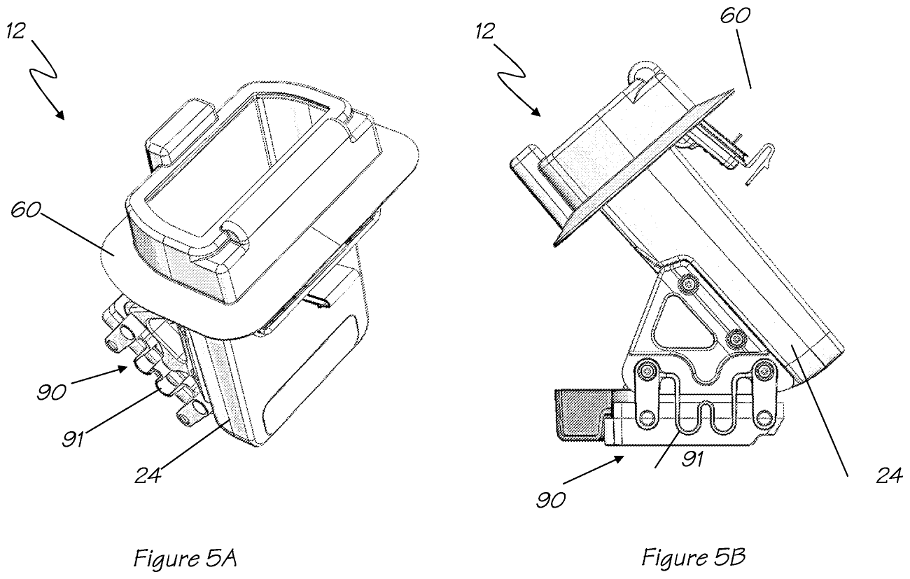

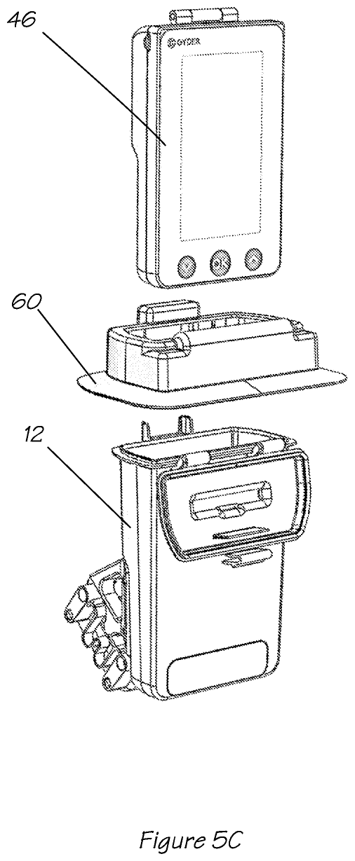

FIGS. 5 to 8 illustrate the electronic orientation monitor 46 and the electronic orientation monitor housing 12. The electronic monitor housing 12 is a sterile housing so that the electronic orientation monitor 46 need not be sterile for use on a patient. In one embodiment, the electronic monitor housing 12 is a clamshell housing. The electronic monitor housing 12 includes a body 24 for receiving the electronic orientation monitor 46, a lid 41, a load funnel 37 and a locking means 80.

The clamshell housing 12 performs three main functions:
1. It provides a physical barrier against biological contamination of the orientation monitor.
2. It provides physical vibration isolation from high levels of shock for the orientation monitor that it would otherwise experience when being used.
3. It maintains proper alignment of the orientation monitor. The accuracy of orientation monitor is highly sensitive to any misalignment so the clam-shell has alignment features built in to maintain proper alignment during use.

The clam-shell housing 12 is preferably designed to be supplied as a sterilised, single use part. Re-using a clam-shell housing 12 can pose a safety risk for the patient through one or more of:
misalignment caused by wear and tear from re-use;
misalignment because of deformation from cleaning or handling;
contamination leading to infection because of incorrect or poor re-sterilisation.

The load funnel 37 includes an angled leading wall 38 and an insertion wall 39. The load funnel is sterile and prevents non-sterile components touching sterile components of the electronic orientation monitor housing 12. The load funnel is inserted into the electronic orientation monitor housing 12 to allow the non-sterile electronic orientation monitor 46 to be inserted into the electronic orientation monitor housing 12. When in the electronic orientation monitor housing 12, the non-sterile electronic orientation monitor 46 can be used in a sterile environment and can withstand forces applied to the electronic orientation monitor housing 12 as discussed further below. Housing 12 also preferably includes one or more internal locating features adapted to ensure the correct alignment of the monitor 46 within housing 12, and thus to ensure repeatable and accurate orientation measurements.

In an alternative embodiment shown in FIG. 5A, load funnel 37 is replaced with a transfer shield 60. Shield 60 is placed over the opening of housing 12 to protect the sterile components of housing 12 whilst monitor 46 is inserted through shield 60 and into housing 12. Shield 60 provides for improved protection to the sterile components of housing 12 during insertion of the monitor 46 than the load funnel 37 of FIG. 5. The housing 12 protects the navigation unit during use to provide impact isolation and a physical barrier to bio-contamination.

The electronic orientation monitor housing 12 includes body 24, cap 41, clip 42 and clip lock 47 arranged to receive and lock the clip 42 and cap 41 in place to seal the body 24. The cap 41, clip 42 and clip lock 47 are part of lid 43 of the electronic orientation monitor housing 12. The cap 41 pivots around a connection with the lid 43 to seal the body 24.

Locking means 80 extends from the base of the electronic orientation monitor housing 12 to engage with the docking stations 19 to fix the electronic orientation monitor 46 to the reference apparatus 100 for obtaining a reference orientation.

The locking means 80 includes perpendicular extension members 25 that extend perpendicularly

from the body 24. There are a forward and rear pair of perpendicular extension members 25 that form a square profile. Support frame member 26 extends between forward and rear perpendicular extension members 25 to form an enclosed frame. The perpendicular extension members 25 are attached to the body at upper pivot points 45. This allows the perpendicular extension members 25 to rotate around upper pivot points 45. The support frame member 26 is attached to the perpendicular extension members using lower pivot points 44. This allows the ends of the perpendicular extension members 25 to pivot around the support frame member 26. The attachment means is further adapted to engage with a mounting frame 90 includes vibration isolation features to absorb forces applied to the electronic orientation monitor housing 12 longitudinally will be described below. Isolation features in particular embodiments include spring features 91 as discussed below and shown, for example, in FIG. 5B.

The enclosed frame of locking means 80 includes a central aperture 48 that is shaped to receive the docking station 19 within it to fix the electronic orientation monitor 46 to the reference apparatus 100 to orient the electric orientation monitor 46 with respect to a patient for further surgical use.

A further embodiment of electronic monitor housing 12 is shown in FIGS. 5A to 5C. In this further embodiment, the angle of the housing 24 is set at an inclined angle by mounting frame 90 to permit easier viewing by the surgeon and surgical staff. Mounting frame 90 is configured to precisely engage with docking stations 19 of the referencing apparatus 100. Spring features 91 of the mounting frame 90 act to isolate the orientation monitor housing 12 and installed orientation monitor 46 from environmental vibrations during operation to improve the positional accuracy able to be provided by the orientation monitor 46.

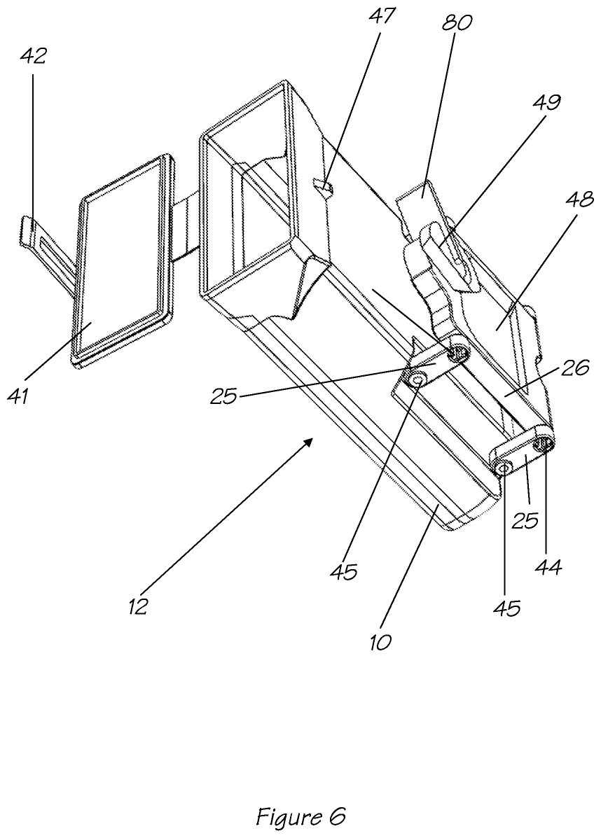

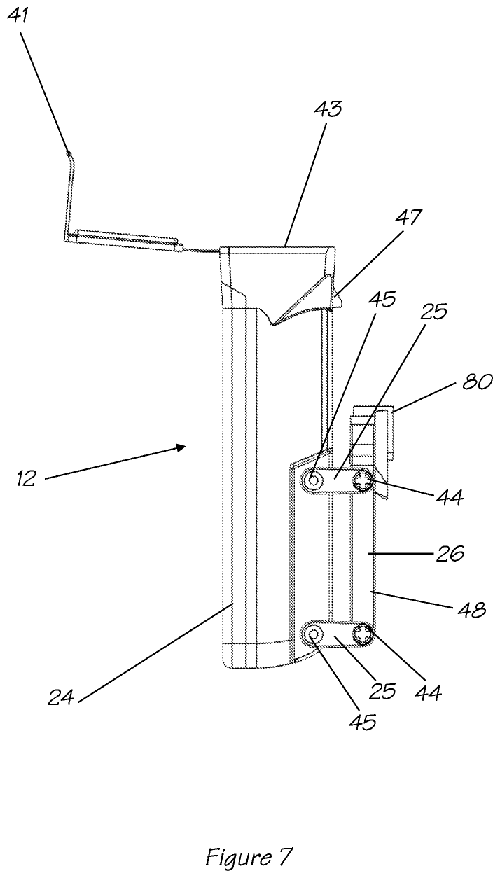

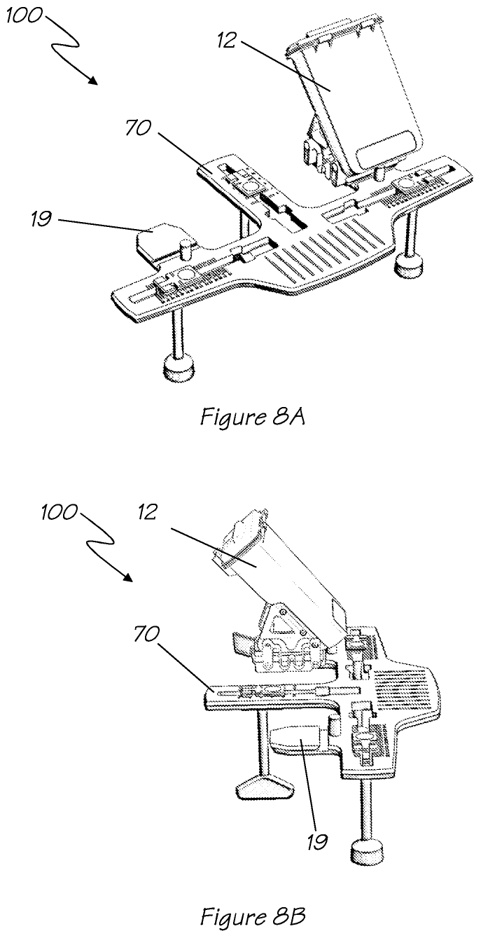

FIGS. 6 and 7 show further views of the clamshell housing 12 disclosed above. FIGS. 8A and 8B depict the clamshell housing 12 engaged with frame 70 of referencing apparatus 100.

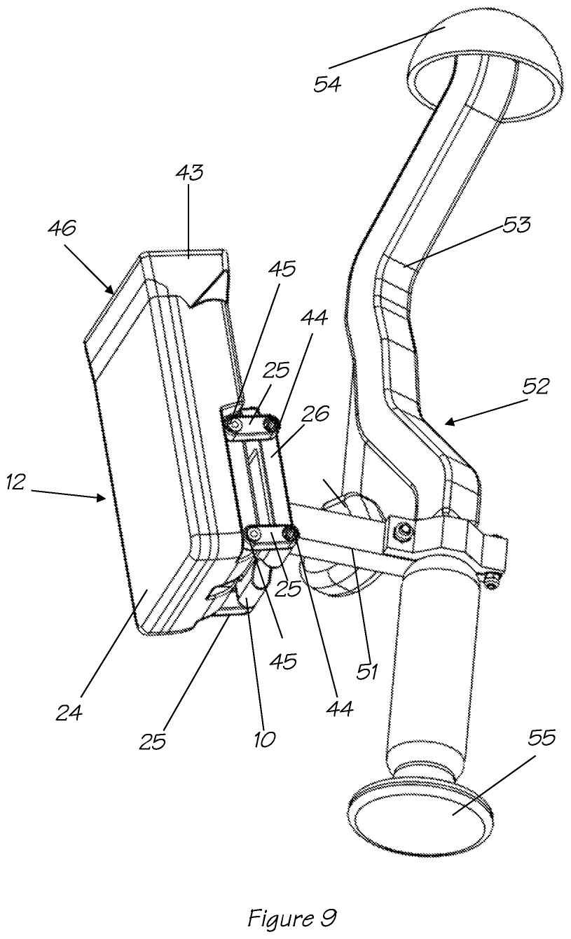

Referring to FIG. 9, with an orientation assumed by the electrical orientation monitor 46, the electronic orientation monitor housing 12 with the electronic orientation monitor 46 inserted is attached to an insertion implement 52 having an acetabular cup 54. The surgeon manipulates the insertion implement 52 into a position whereby the cup is adjacent the acetabulum of the patient and a display (not shown) on the electronic orientation monitor 46 guides the surgeon to orient the insertion implement such that the current orientation of the electronic orientation monitor 46 is equal to the reference orientation (or such that the current orientation has some other desired relationship to the reference orientation). The display provides positional and orientational information to the surgeon including: yaw/inclination axis orientation and anteversion/pitch axis orientation to allow the surgeon to orient the prosthetic tool precisely with the patient's unique anatomical structure. The orientation monitor 46 may provide orientation information to the surgeon in a number of different ways, depending on the particular surgeon's preference. For example the orientation of the insertion implement 52 with respect to the patient's acetabulum may be conveyed to the surgeon through a simple target display, where the degree the implement 56 is off-target, may be conveyed visually. Further examples of an orientation display may include a “bubble-level” visual display, or a numerical display depending upon the surgeon's personal preference.

Once the desired orientation has been achieved, the electronic orientation monitor 46 provides an indication to the surgeon, such as a visual indication on the display and/or an audible indication, and this prompts the surgeon to either assess the trial cup, or to hit the impactor plate 55 to transfer force through the shaft 53 to impact the acetabular cup 54 into the patient's reamed acetabulum.

When placed on the insertion implement 52, the electrical orientation monitor housing 12 is arranged to isolate shock from the electronic orientation monitor 46. The perpendicular extension members 25 pivot around pivot points 44 and 45 to move the support frame member 26 forwards or backwards to isolate force applied to the impactor plate 55 from the electronic orientation monitor 46. Spring formations 91 of the embodiment of housing 12 as seen in FIG. 5B are particularly adapted for shock isolation from the insertion implement during use.

From the foregoing description it will be appreciated that the referencing apparatus 100 is configured for solely anterior engagement with the patient and the patient connector plates 18 that are on the patient during the steps of pressing the referencing apparatus into engagement with the surgical drapes and during calibration of the electronic orientation monitor. This compares favourably with the clamp disclosed in WO 2010/031111, which requires both anterior and posterior engagement with the patient. This is because it has been appreciated by the present inventor that in practice the clamp of WO 2010/031111 may cause an undesirable obstruction to the surgeon.

Whilst the invention has been described with reference to specific examples, those skilled in the art will appreciate that it may be embodied in many other forms.

The Claims defining the invention are as follows:

1. A referencing apparatus for application onto a patient, the referencing apparatus including:
a support plate;
a first locating element extending from the support plate;
a second locating element extending from the upper support plate;
a third locating element extending from the upper support plate; and
an electronic orientation monitor housing adapted to receive an electronic orientation monitor;
a docking station adapted to receive the housing and thereby define a reference point external to and relative to the patient's anatomy;
wherein the first locating element, the second locating element and the third locating element are arranged to be pushed down onto a patient to provide a reference orientation for the electronic orientation monitor with respect to a predetermined anatomical site.

2. A referencing apparatus according to claim 1, wherein the housing is a sterile housing.

3. A referencing apparatus according to claim 2, wherein the housing comprises:
a body comprising an opening adapted for receiving the orientation monitor;
a load funnel comprising an angled, leading wall, and an insertion wall for enabling a non-sterile orientation monitor to be inserted into the sterile housing whilst preventing contamination of the sterile housing; and
a locking means comprising an enclosed frame adapted for engagement with the docking station, wherein the frame is pivotally attached to the body of the housing.

4. A referencing apparatus according to claim 3, wherein the load funnel is a transfer shield adapted to be placed over the opening of the housing to protect the sterile components of housing in use whilst the monitor is inserted through the shield and into the housing.

5. A referencing apparatus according to claim 3, wherein the locking means comprises a central aperture shaped to receive the docking station within it, thereby to fix the orientation monitor to the reference apparatus to orient the electric orientation monitor with respect to the patient.

6. A referencing apparatus according to claim 1, wherein the housing is a clamshell housing.

7. A referencing apparatus according to claim 1, wherein the first and second locating elements are first and second anterior superior iliac spine locating elements.

8. A referencing apparatus according to claim 1, wherein the third locating element is a pubis locating element.

9. A referencing apparatus according to claim 1, wherein the first and second locating elements include a patient connecting plate.

8. A referencing apparatus according to claim 1, wherein the patient connecting plate is arranged to fix to a patient above the anterior superior iliac spine.

11. A referencing apparatus according to claim 1, wherein the electronic orientation monitor is housed in an electronic orientation monitor housing.

12. A referencing apparatus according to either 1, wherein the electronic orientation monitor housing is arranged to engage the support plate to provide the reference orientation for the electronic orientation monitor.

13. A referencing apparatus according to claim 1, wherein an electronic orientation monitor is dockable with the docking station in either a first docking configuration or a second docking configuration.

14. A referencing apparatus according to claim 13:
wherein the first docking configuration defines a first orientation of the electronic orientation monitor relative to the referencing apparatus; and
wherein the second docking configuration defines a second orientation of the electronic orientation monitor relative to the referencing apparatus.

15. A method of using a referencing apparatus as claimed in claim 1, to calibrate an electronic orientation monitor, the method including the steps of:
positioning a patient connector plate over the left anterior superior iliac spine;
positioning a patient connector plate over the right anterior superior iliac spine;
connecting the first and second locating elements to respective patient connector plates;
positioning the third locating element against the pubis;
placing an electronic orientation monitor into a sterile housing comprising means for receiving the non-sterile monitor whilst preventing contamination with the sterile housing;
engaging the housing comprising the monitor housed therein to a docking station disposed on the referencing apparatus to assume a reference orientation external to and relative to the patient; and
calibrating the electronic orientation monitor.

16. A method according to claim 1, wherein the referencing apparatus is disposed anterior to the patient during the steps of pressing the referencing apparatus into engagement with the surgical drapes and during calibration of the electronic orientation monitor.

17. A method according to claim 15, wherein an anterior approach is used for surgical access to the patent's acetabulum.

18. A method according to claim 15, wherein the patient lies in a face up position during said steps.

19. A method of using a referencing apparatus as claimed in claim 1, to calibrate an electronic orientation monitor, the method including the steps of:
positioning surgical drapes onto a patient;
engaging the plurality of locating elements against the surgical drapes such that the surgical drapes are disposed intermediate the plurality of locating elements and a respective plurality of predefined anatomical sites on the patient so as to cause a docking station disposed on the referencing apparatus to assume a reference orientation relative to the plurality of predefined anatomical sites;
placing an electronic orientation monitor into a sterile housing comprising means for receiving the non-sterile monitor whilst preventing contamination with the sterile housing and docking the housing with the docking station so as to orient the electronic orientation monitor in a reference orientation; and
calibrating the electronic orientation monitor.
